(19) United States
(12) Patent Application Publication
(10) Pub. No.: US 2008/0241841 A1
(43) Pub. Date: Oct. 2, 2008

(54) Method and apparatus for sample preparation

(75) Inventors: Katsuji Murakawa, Kodaira (JP); Sumiyo Takiguchi, Koganei (JP); Hideki Kambara, Hachioji (JP)

Correspondence Address: Stanley P. Fisher;Reed Smith LLP, 3110 Fairview Park Drive, Suite 1400, Falls Church, VA, 22042-4503, US

(73) Assignee: Hitachi, Ltd.

(21) Appl. No.: 11/984,065

(22) Filed: Nov. 13, 2007

(30) Foreign Application Priority Data
Mar. 30, 2007 (JP) 2007-093618

(51) Int. Cl. C12Q 1/68; C12P 21/04; C12M 1/34

(52) U.S. Cl. 435/6; 435/91.2; 435/287.2

(57) ABSTRACT

A method of the present invention comprises fractionating a sample solution containing analyte DNA molecules into small droplets, wherein the number M of the droplets is greater than the total number N of the DNA molecules, subjecting an emulsion containing the droplets to, for example, PCR amplification, and detecting the presence or absence (amount) of an amplicon obtained in each droplet by fluorescent detection using an intercalator or the like.

### CLAIM OF PRIORITY

The present application claims priority from Japanese application JP 2007-093618 filed on Mar. 30, 2007, the content of which is hereby incorporated by reference into this application.

### BACKGROUND OF THE INVENTION

1. Field of the Invention

The present invention relates to a method for sample preparation for gene analysis techniques. More specifically, the present invention relates to a method for sample preparation for digital analysis of messenger RNAs (mRNAs) contained in one cell or for a method for analyzing a large number of target molecules simultaneously and individually.

2. Background Art

As the complete human genome sequence has been unveiled, the time has come when various genomic information has been examined energetically and exploited. Genomic information is transcribed to mRNAs and translated to proteins. Such gene expression profiling analysis is essential to examine details of life activity. Conventional mainstream analysis methods involved isolating mRNAs from many cells, fluorescently labeling the mRNAs, allowing them to act on a DNA probe array (DNA chip), and capturing the labeled mRNAs for detection by probes having complementary sequences to mRNAs. By contrast, another method involves isolating mRNAs from many cells, preparing complementary DNAs (cDNAs) thereof, and electrophoretically separating them for measurement. This method measures the amounts of a variety of mRNAs in an analog fashion and however, must take out mRNAs from many cells for measurement in terms of measurement sensitivity problems.

On the other hand, many cells constitute one system in coordination to maintain life activity. Individual cells in tissue have been thought to play their respective different roles. For understanding actual life, it is important to monitor the roles of such individual cells. Thus, the measurement of mRNAs or proteins contained in one cell is beginning to be valued. This measurement requires accurately quantitatively analyzing the types and amounts of mRNAs contained in small amounts in one cell. However, such methods have not been established so far.

To overcome this problem, the present inventors are aiming to conduct quantitative analysis by the digital counting of all mRNAs contained in one cell or a plurality of mRNAs probably in need of measurement. The digital counting is a method for quantitative analysis by determining the type of each mRNA (or cDNA fragment) by sequencing and counting the number of mRNAs with this sequence contained in the cell.

Specifically, the digital counting is performed by analyzing the sequence of each of plural mRNAs or DNA fragments contained in a small region such as a cell. This technique requires individually amplifying individual mRNAs (or cDNA fragments) and analyzing their sequences. What is important here is to amplify all mRNAs (or cDNA fragments) each independently and completely.

In the method described above, many PCR amplifications are performed in parallel with one DNA or mRNA molecule as a starting material. A sample used in this method is in a solution state and contains mRNAs or cDNA fragments on the order of several tens to several millions. The PCR amplification of these mRNAs or cDNA fragments by one operation merely produces a mixture of plural amplicons and does not provide expected measurement samples. Thus, the method requires amplifying individual mRNAs each independently and completely and isolating them separately. To amplify individual mRNAs each independently, they are individually amplified in a separated state by PCR. This PCR requires diluting and fractionating a sample solution so that the expected number of DNA or RNA molecules per reaction volume is one or less at the start of reaction, and amplifying these fractions each independently by PCR. For example, when the number of molecules to be amplified in a certain sample is expected to be 100,000, a sample solution is diluted and fractionated to hundreds of thousands of fractions. These fractions can be amplified each individually by PCR (polymerase chain reaction) or the like to thereby amplify all the molecules in the sample each independently, that is, to thereby achieve clone amplification.

Several attempts have been made in recent years to individually amplify plural DNAs by such a method. For example, a very large number of small reaction cells are provided on a flat plate. A solution containing target DNA fragments and enzymes and reaction substrates necessary for amplification is poured onto the plate and fractionated to the small reaction cells. The fractionated PCR solutions are mutually separated and can therefore be amplified each independently. The individual amplification is achieved by adjusting the amount (i.e., number) of the DNA sample contained in one fraction to one or less in average. One example of this method has been disclosed in Analytical Chemistry (Anal. Chem. 2001, 73, p 1043-1047). In this example, 10,000 wells (microchambers) are constructed on a silicon substrate for high degree of integration. However, the amplification of 1,000,000 DNA fragments requires a larger number of reaction cells. Moreover, it is impossible to exhaustively inject the whole target sample solution into small reaction cells. In some cases, a certain amount of the sample solution is left over, or otherwise, DNAs are adsorbed onto the inner walls of reaction cells. Thus, some DNAs are not used in PCR amplification.

Alternatively, for example, PCR is performed using not a microtiter plate but a gel dot matrix arranged in a plane (JP Patent Publication (Kokai) No. 2004-337064A (2004)), though this attempt does not intend amplification from one molecule. A method has heretofore been known in the art, which comprises gelling, for improvement in sample handleability, a PCR product sample solution with a material that is gelled at low temperatures (JP Patent Publication (Kokai) No. 10-004963A (1998)). In this example, a chip for genetic testing in which the gelled sample is arranged in a matrix form is used. However, this method uses spatially fixed reaction cells, some of which thus contain an expected amplicon but the others of which contain no amplicon. Therefore, some target samples are unamplified. Thus, the problem of this method is how to select the expected amplicon.

Another effective method is called emulsion PCR. In this method, reaction is performed in a large number of small droplets formed in oil, instead of using independent reaction vessels on a sample-by-sample basis. In this method, small droplet formation is easily achieved by stirring or the like. Therefore, droplets equal to or more than hundreds of thousands of reaction vessels can be formed in one vessel of approximately 100 microliters.

However, in the method using an emulsion, it is not easy to individually collect samples from individual droplets. Therefore, DNAs or RNAs are immobilized in droplets, and beads bound with a probe are added to a reaction solution. The DNA or RNA in each droplet is collected by separating the beads capturing the formed reaction product from the solution. Such sample collection using bead solid phases requires separately collecting a solid phase with a product and a solid phase with no product for collecting DNAs or RNAs obtained from enzyme reaction or the like. Therefore, a method has been used, which comprises preparing magnetic beads in which probes having a sequence complementary to a portion of DNA obtained by PCR are immobilized, hybridizing the probes to the amplified DNA fragments, and selecting and collecting the DNA fragments with a magnet. An example of amplification and genome sequencing of many DNA fragments using this method has been published in, for example, Nature (WO2005/10145 (PCT/US2004/015587) and Nature. 2005, 437, p 376-380, (Supplementary Information)). However, this technique, when applied to the amplification and sequencing of all mRNAs, presents a serious problem as expected. In this system, a bead and one copy of target DNA must be contained in one reaction droplet in an emulsion. If two or more beads are contained in the formed droplet, one mRNA is doubly counted. Therefore, digital counting cannot be used in this technique. To solve this problem, the amount of beads may be reduced to a level almost equal to that of DNAs. However, in such a case, a large number of droplets contain DNA but no bead. Therefore, this approach is also inconvenient. The collection of produced DNAs with solid beads is a good method, and this method is sufficiently available for genomic sequencing using overlapping DNA samples and however, is unsuitable for digital counting.

All the conventional methods had problems, as described above. First, the technique using a microtiter plate does not give consideration to liquid handling during the isolation of amplicons derived from a large number of simultaneously treated samples. A large number of samples are individually collected in a liquid state by distinguishing the amplified reaction products. Therefore, this technique had the problems of many sample vessels required according to the number of the samples and complicated handling procedures.

The method comprising capturing amplicons by bead surface and collecting them requires immobilizing in advance primers or the like necessary for reaction onto the beads. This method had the problem of reduction in amplification efficiency for obtaining amplicons on the solid surface using the primers immobilized on the solid phases as amplification primers. This is because the degree of freedom of motion of DNA or RNA molecules as enzyme reaction substrates is lowered due to immobilization thereof, resulting in largely reduced reaction efficiency compared to solution systems. Furthermore, this method had the problem of non-specific adsorption of DNAs or RNAs to solid phase surface. Specifically, DNA fragments as initial amplification templates do not well work, when adsorbed to the solid phase. As a result, one copy of the DNA template is contained in an emulsion. However, no amplicon is obtained. Particularly, when DNA or RNA samples with such an exceedingly low concentration as one molecule per reaction solution are used as starting materials for clone amplification, the influence of non-specific adsorption is relatively large and becomes a serious problem. Furthermore, it is difficult to uniformly inject beads to individual reaction solutions in a droplet emulsion form, as described above. Particularly, when an emulsion is prepared by stirring, it is impossible to inject the same numbers of solid phases such as beads to all droplets. In this case, one droplet contains plural beads or contains no beads. If the number of solid phases such as beads per droplet cannot be controlled, it is difficult to precisely perform single molecule measurement aimed at all molecules in a sample.

Thus, none of the conventional methods were suitable for the purpose of simultaneously amplifying and collecting all components constituting a DNA fragment pool (population of mRNAs or cDNA fragments obtained from one cell) as a sample.

The present invention has been completed for overcoming such problems of the conventional techniques. An object of the present invention is to prepare DNA sequencing samples by isolating mRNAs contained in one cell, reverse-transcribing these mRNAs to cDNAs, performing amplification on a molecule-by-molecule basis, and collecting them. Specifically, an object of the present invention is to provide a technique for amplifying, on a molecule-by-molecule basis, all components contained in a DNA fragment pool by a convenient method and individually collecting them.

### SUMMARY OF THE INVENTION

The present inventors have conducted diligent studies for attaining the object and have devised a method of reliably achieving amplification on a molecule-by-molecule basis and isolating only the amplified reaction product. As a result, the present inventors have succeeded in amplifying, on a molecule-by-molecule basis, all mRNAs (cDNAs) contained in one cell and individually collecting them.

Specifically, the present invention relates to a method for individually amplifying and isolating a plurality of nucleic acids in a sample, comprising subjecting the sample diluted so that the number of the nucleic acid contained in one droplet does not exceed one to PCR in the droplets in a hydrophobic solution and separating the reaction solution in a solid or gel state after the completion of PCR.

The method may further comprise the step of adding in advance a fluorophore capable of binding to or intercalating into an amplicon to the PCR reaction solution and thereby selecting and separating only the droplet containing the amplicon. Examples of such a fluorophore can include an intercalator and a fluorescently labeled molecular beacon.

It is desired that an adaptor sequence should be introduced in advance in each of a plurality of nucleic acids in a sample so as to amplify a plurality of nucleic acids with a single PCR primer.

In the present invention, the droplets are each independently amplified. Therefore, it is desired that the PCR should be performed in an emulsion of the droplets dispersed in the hydrophobic solution or in mutually separated small reaction cells arranged in a plate.

A gelling agent for forming a hydrogel selected from water-soluble synthetic polymers such as agarose, gelatin, starch (amylose), carrageenan, pectin, agaropectin, polyacrylamide, polyacrylic acid, polyvinyl alcohol, and polyvinylpyrrolidone is added in advance to the PCR reaction solution for separating the reaction solution in a solid or gel state.

It is preferred that the hydrophobic solution used in the present invention should mainly be composed of silicone oil or paraffin oil.

Moreover, it is preferred that a surfactant (e.g., amphiphiles) and/or a coating agent should be added in advance to the PCR reaction solution for improving droplet stability in the hydrophobic solution.

The present invention also provides a method for nucleic acid analysis comprising the step of detecting or quantifying a plurality of nucleic acids individually amplified and isolated by the method.

The present invention further provides an apparatus used in the method, comprising: 1) a sample handling device comprising a temperature control device for storing a gelling agent in a solution state, a liquid handling device for mixing the gelling agent and a reaction solution, and a stirring device; 2) a droplet formation device comprising any of an oscillating or rotating mixer, an ink jet, and microfluidics; 3) a temperature control device having a thermal cycle function for PCR; and 4) a fluorescent detection device equipped with an imaging or flow-cell detector.

In the apparatus, it is desired that the flow cell in the fluorescent detection device 4) should have a separation function by channel switching.

The present invention further provides a system for nucleic acid analysis comprising the apparatus and a DNA sequencer and/or a flow cytometry.

According to the present invention, a large number of samples in small amounts such as all mRNAs contained in one cell can be amplified simultaneously and individually by PCR, and the obtained amplicons can be identified on the basis of fluorescence and collected as gelled droplets. This collection does not require providing a solid phase in a reaction solution. Therefore, cost and labors for this purpose are saved. Moreover, a sample loss and reduction in reaction efficiency attributed to a solid phase can be prevented.

### BRIEF DESCRIPTION OF THE DRAWINGS

FIG. 1 is a schematic diagram of a method of the present invention;

FIG. 2 is a schematic diagram of the method of the present invention applied to cDNAs;

FIG. 3 is a flow chart of the method of the present invention;

FIG. 4 is data of Example 1 of the present invention;

FIG. 5 is an illustrative diagram of Example 1 of the present invention;

FIG. 6 is data of Example 1 of the present invention;

FIG. 7 is data of Example 1 of the present invention;

FIG. 8 is an illustrative diagram of Example 2 of the present invention;

FIG. 9 is an illustrative diagram of Example 2 of the present invention;

FIG. 10 is an illustrative diagram of Example 2 of the present invention;

FIG. 11 is an illustrative diagram of Example 3 of the present invention;

FIG. 12 is an illustrative diagram of Example 4 of the present invention; and

FIG. 13 is an illustrative diagram of Example 5 of the present invention.

### DETAILED DESCRIPTION OF THE PREFERRED EMBODIMENTS

In the present invention, a large number of PCR amplifications in small droplets are simultaneously performed using a reaction solution in an emulsion state without adding solid beads, which are factors inhibiting amplification, to PCR or without using small reaction cells made of solid matters. Then, only the reaction solution containing the synthesized complementary DNA strand is collected.

In the present invention, “(small) droplets” refer to fine droplets, wherein one droplet is capable of containing one nucleic acid. The size of the droplets is not particularly limited and is, preferably, approximately 1 μm to 150 μm in diameter. Moreover, a “small cell” refers to a cell for accommodating one of the droplets. The size of the small cell is not particularly limited. It is preferred that 100,000 or more small cells of approximately 3 μm to 250 μm in diameter should be provided.

A sample is sufficiently diluted for use so that the number of the nucleic acid contained in one of the droplets does not exceed one. Moreover, an adaptor sequence is introduced in advance in each of the nucleic acids in a sample so as to amplify the nucleic acids with a single PCR primer. The adaptor sequence can be introduced by a method known in the art, for example, by using a primer containing the adaptor sequence during cDNA synthesis from mRNAs.

The PCR is performed in a solution state in the absence of solid phases such as beads and thereby allowed to efficiently proceed. Next, the emulsion containing an amplicon is cooled and isolated in a solid or gel state. PCR is usually performed at a high temperature of 50 to 96° C. Therefore, the emulsion can be isolated in a solid or gel state at room temperature or lower temperatures. Specifically, this is achieved by adding, to the reaction solution, a substance that is liquid at high temperatures and solid or gelled at low temperatures.

There exist a variety of methods for distinguishing whether complementary strand synthesis is accomplished or not. In Examples of the present invention, fluorescent detection using an intercalator that emits fluorescence through intercalation into double-stranded DNA is illustrated as an example. Examples of the intercalator can include SYBR Green I, PicoGreen, and ethidium bromide. The detection is not limited to the method using the intercalator. A probe that emits fluorescence upon complementary strand synthesis, such as a molecular beacon may be used.

The isolated gel or reaction solution beads (thus called because of the solid or gel state) are irradiated with a laser. Those emitting fluorescence are selected and captured. In this procedure, an existing apparatus such as a flow cytometry can be used. In addition, for example, a bead selector using microfluidics can be utilized.

In the method, a material that can be used for isolating the solidified or gelled reaction products is a hydrophilic gelling agent such as agarose, gelatin, starch (amylose), or polyacrylamide. These gelling agents are soluble by heat and can therefore achieve reaction in a solution system with good reaction efficiency. Specifically, aqueous solutions of these gelling agents are in a solution state under conditions of 50° C. or higher (reaction temperatures of general thermostable enzymes) and is gelled under conditions of room temperature during the isolation of reaction products. When the reaction solution must be in a solution state at approximately 37° C., low melting agarose may be used. In addition, a substance that is rendered solid by complementary DNA strand synthesis may be added, as a matter of course.

### EXAMPLES

Hereinafter, the present invention will be described in detail with reference to Examples. However, the present invention is not intended to be limited to these Examples.

### Example 1

In this Example, the preparation of an emulsion containing agarose by stirring in oil is illustrated as an example.

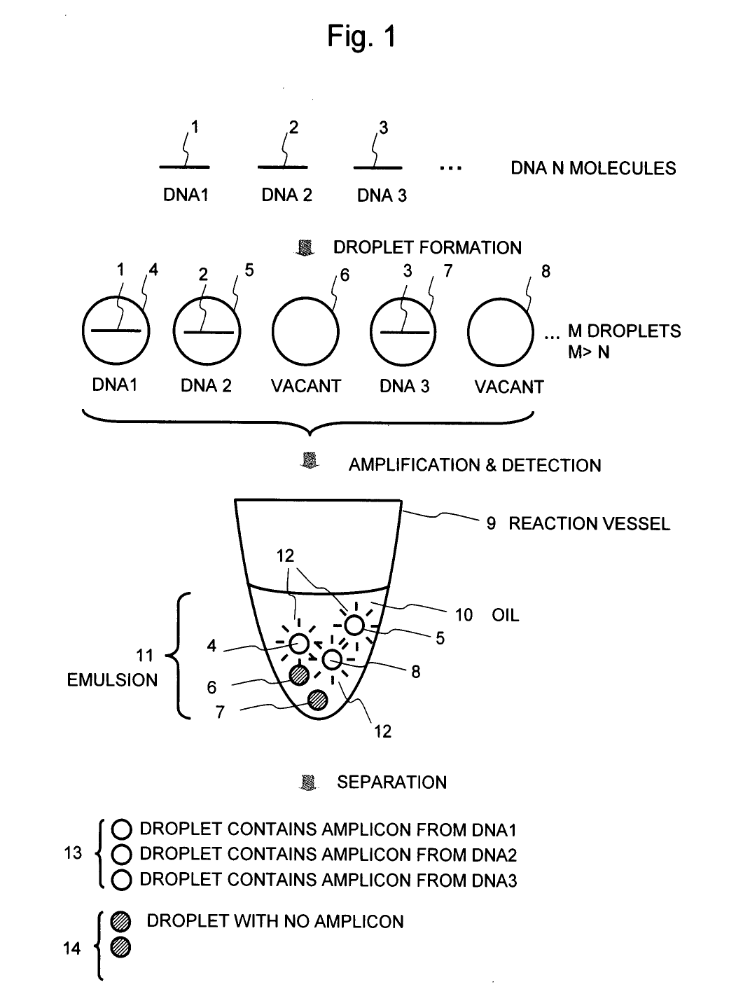

FIG. 1 shows the basic concept of the present method. A sample solution containing analyte DNA molecules 1 to 3 is fractionated into small droplets 4 to 8, wherein the number M of the droplets is greater than the total number N of the DNA molecules. As a result, the droplets 4, 5, and 7 containing the DNA and the droplets 6 and 8 containing no DNA are formed. The droplets 4 to 8 are dispersed into oil 10 in a reaction vessel 9 to form an emulsion 11. This emulsion containing the droplets is subjected to, for example, PCR amplification. Then, the presence or absence (amount) of an amplicon obtained in each droplet is detected by fluorescent detection using an intercalator or the like to make a separation between a droplet 13 that contains an amplicon from each of the DNAs 1 to 3, from which fluorescence 12 is detected, and a droplet 14 with no amplicon, from which fluorescence is not detected. A gelling agent that is gelled or solid at room temperature can be contained in advance in the droplets to thereby separate the individual droplets. Specifically, the expected amplicon can be obtained by collecting only the droplet (gel) that emits light through laser irradiation or by dissolving and removing the droplet (gel) that does not emit light.

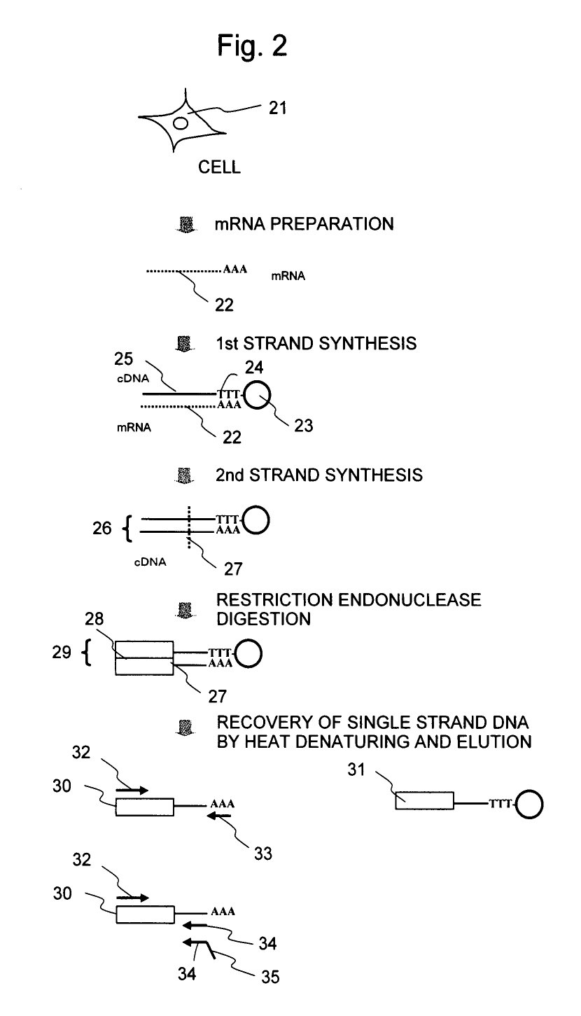

Next, the method of the present invention applied to cDNAs derived from one cell will be described with reference to FIG. 2. mRNA 22 obtained from one cell 21 is captured with a poly(T) oligomer 24 as a probe immobilized on a magnetic bead 23. A complementary DNA strand 25 is synthesized with reverse transcriptase (1st strand synthesis). The mRNA 22 is digested with RNase H. Then, double-stranded cDNA 26 is formed with random primers (2nd strand synthesis). Subsequently, the double-stranded DNA is digested in a sequence-specific manner with restriction endonuclease such as MboI. An adaptor sequence 28 with a known sequence is ligated to a cutting site 27 to create a PCR priming site. A solution containing the thus-obtained double-stranded DNA fragment 29 immobilized on the bead is heated to melt its double strands. Free single-stranded DNA 30 is obtained from a single strand 31 immobilized on the bead 23. Sequences at both ends of this single-stranded DNA are the known sequence of the adaptor sequence 28 at the 5′ end and poly(A) at the 3′ end. Therefore, the adaptor sequence 28 and poly(T) primers can be used in common for PCR amplification. The free single-stranded DNA 30, two primers 32 and 33, and complementary strand synthesis substrates and enzymes were subjected together to PCR amplification in the droplets shown in FIG. 1. In this procedure, agarose that is gelled at low temperatures and an intercalator are added thereto in advance, as described above. PCR, details of which will be described later, is performed by thermal cycles at approximately 50 to 96° C. In this temperature range, the agarose is in a liquid state. After the completion of PCR, the reaction solution containing the agarose is cooled to room temperature and collected as gel beads. On the other hand, to count plural mRNAs having a particular sequence, primers 34 having a sequence specific to their sequences are used. Alternatively, a portion having an unspecific priming sequence 35 is anchored in the primer, and this anchor site may be used as a PCR amplification primer.

In this Example, a model sample was used in the experiment to clearly show the number of DNA templates added. However, similar primers can also be used for individual amplification in actual cDNA measurement.

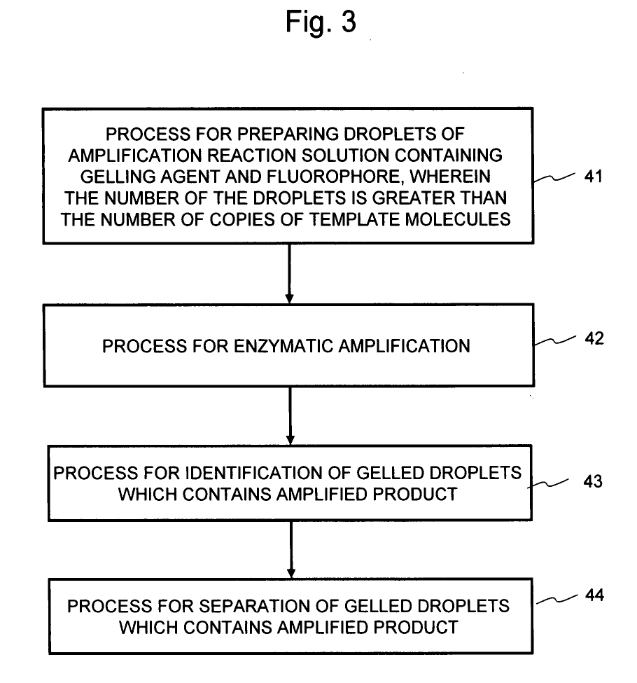

Hereinafter, amplification processes will be described with reference to FIG. 3. The present amplification processes comprise: (1) a process 41 for preparing droplets of an amplification reaction solution containing a gelling agent and a fluorophore in a hydrophobic solution in the same reaction vessel, wherein the number of the droplets is greater than the number of copies of template molecules; (2) a process 42 for enzymatic amplification; (3) a process 43 for identification of the gelled droplets which contain an amplicon; and (4) a process 44 for separation of the gelled droplets which contain an amplicon. Hereinafter, these four processes will be described in detail.

(1) Process for preparing droplets of amplification reaction solution containing gelling agent and fluorophore in hydrophobic solution in same reaction vessel, wherein the number of the droplets is greater than the number of copies of template molecules:

A PCR reaction solution (50 μL/reaction) is prepared according to the following composition: 120 mM Tris-SO₄ (pH 8.9), 36 mM Ammonium Sulfate, 4 mM MgSO₄, 0.4 mM dNTPs, 0.4 μM F primer (GTTTTCCCAGTCACGACGTTG: SEQ ID NO:1), 0.4 μM R primer (ATGACCATGATTACGCCAAGC: SEQ ID NO: 2), and 0.04 unit/μL amplification enzyme Platinum Taq DNA polymerase High Fidelity (Invitrogen).

Template DNA used in the reaction solution was commercially available pUC19 plasmid DNA (2686 bp, Takara Bio) for which a copy number can be estimated. In actuality, the reaction solution containing 10⁴ to 10⁸ molecules/reaction of this template was prepared for confirming amplification efficiency and so on. The number of the plasmid DNA molecules was determined from the concentration (0.5 μg/μl, 1.7×10¹¹ molecules/μl) of the stock solution described in the document attached to the product. Moreover, a SYBR Green I solution (Invitrogen, S7563) was added as a dye for fluorescent detection of PCR products at a 2500-fold dilution of the stock solution to the reaction solution. The molar concentration of this product is not disclosed. Therefore, the dilution is not an absolute numeric value.

In addition to the SYBR Green I, an intercalator whose fluorescent intensity is increased by binding to double-stranded DNA, such as PicoGreen or ethidium bromide may be used as a fluorophore. In addition, a probe that emits fluorescence upon complementary strand synthesis, such as a molecular beacon may be used.

The gelling agent used was agarose. The agarose used was Seakem Gold Agarose (Takara Bio) with high gel strength of 1800 g/cm² (1% (w/v) gel) or more.

A preferable gel concentration is 1 to 1.5% (w/v) for agarose in consideration of both easy liquid handling during reaction setup and hardness required for gel handling during isolation. However, gel strength largely differs among products even if the products are the same gel materials. Therefore, the optimal concentration differs from material to material. To secure gel hardness after isolation or a dry product size after moisture removal from the gel, a gel with a higher concentration may be used. Up to 2.5% (w/v) agarose and up to 5.0% (w/v) gelatin can work in PCR without any major difficulties.

Agarose powders are difficult to dissolve. Therefore, the agarose is heated in advance to 121° C. with an autoclave to prepare a uniform aqueous solution of 2.5% (w/v) agarose with a temperature of 50° C. or higher at which the agarose has a viscosity that permits easy pipetting. This aqueous solution of 2.5% agarose is quickly mixed with the PCR reaction solution set to approximately 50° C. in equal volumes (50 μl/reaction) to prepare a reaction solution (100 μl in total/reaction) with a final agarose concentration of 1.25% (w/v). The mixing is performed at a temperature of 90° C. or lower, which does not influence thermostable enzymes.

The oil used for emulsion preparation was silicone mixed oil. Its composition was as follows with reference to the description of the document (Nature, 2005, 437, p 376-380, (Supplementary Information)): (1) 25% (v/v) Polyphenylmethylsiloxane (Fluka, trade name: AR20), (2) 10% (v/v) PEG/PPG-18/18 Dimethicone polymer, 50% (v/v) Decamethylpentacyclosiloxane solution (Dow Corning Toray, trade name: DC5225C), and (3) 50% (v/v) Trimethylsiloxysilicate, 25% (v/v) Decamethylpentacyclosiloxane solution (Dow Corning Toray, trade name: BY11-018).

Specifically, these components are Polyphenylmethylsiloxane serving as base oil, Decamethylpentacyclosiloxane serving as a solvent, PEG/PPG-18/18 Dimethicone serving as a polymer with surfactant effects and viscosity, and Trimethylsiloxysilicate serving as a component for forming a silicate coating in an interface to water.

This mixed oil is mixed with the gelling agent-containing reaction solution in equal amounts (100 μl/reaction) to prepare an emulsion (200 μl/reaction after mixing). The mixed solution is added to a 2-ml sample tube and stirred for approximately 2 to 5 seconds with a vortex mixer (Taitec, 2500 rpm) to obtain small droplets of approximately 50 to 100 μm in diameter.

The size of the droplets may be changed according to an expected amplification factor and the number of copies of template molecules and is preferably 20 to 200 μm in diameter. Particularly, droplets of approximately 50 to 100 μm in diameter are preferable for amplifying approximately 100,000 molecules corresponding to the number of genes present in one cell. In this size range, a sufficient amount of reagent components necessary for the amplification is secured, while the total amount of the reaction solution is 1 ml or smaller, which permits easy handling.

A method for forming the droplet emulsion of the reaction solution is not particularly limited. In addition to the stirring with a mixer, an ink jet method, a method using microfluidics (Angew. Chem. Int. Ed. 2005, 44, p 724-728), and so on may be used.

The obtained emulsion may be amplified in a general plastic reaction vessel. The amplification may be performed in, in addition to the general reaction vessel, mutually separated small reaction cells arranged in a plate for the purpose of simplifying observation after reaction.

Changes in the mixing ratio of the components in the oil do not largely influence the formation of droplets themselves of the reaction solution and however, influence emulsion stability to a certain extent. Oil made of 100% Polyphenylmethylsiloxane as base oil is particularly preferable for optical detection, because the oil portion is not opaque and is clear even after emulsion formation. In this case, the droplets of the reaction solution tend to aggregate. However, the droplets do not fuse into one mass by virtue of the gelling agent contained in the reaction solution. When Trimethylsiloxysilicate is added in a component amount of approximately 5% (1/10 volume in 50% solution) or more to Polyphenylmethylsiloxane as base oil, the aggregation of the droplets is eliminated. Trimethylsiloxysilicate added in a component amount increased to 25% (1/2 volume in 50% solution) produces the same effects.

When PEG/PPG-18/18 is added in a component amount of 1% (v/v) (1/10 volume in 10% solution) or more to Polyphenylmethylsiloxane as base oil, the formed emulsion is entirely opaque. In this case, the separation between the droplet and the oil in the emulsion is suppressed, resulting in improved emulsion stability. PEG/PPG-18/18 added in a component amount increased even to 7% (v/v) (7/10 volume in 10% solution) produces almost the same effects.

The surfactant, the thickener, and the coating agent used above may be substituted by analogous substances.

In addition to the silicone (organosilicon) oil, paraffin oil such as mineral oil may be used as a hydrophobic solution. The silicone oil has a density of approximately 0.98, which is close to the density (1) of water serving as a solvent of the reaction solution. Furthermore, the viscosity of the silicone oil is hardly changed due to a temperature. Thus, the silicone oil permits stable emulsion formation with the reaction solution and is therefore particularly preferable.

(2) Process for Enzymatic Amplification

The prepared reaction solution in an emulsion state is dispensed in 50 μl aliquots to 0.2-ml tubes and subjected to PCR amplification under thermal cycle conditions involving 94° C. for 15 seconds, 55° C. for 30 seconds, and 70° C. for 1 minute. The number of cycles is 40 cycles. Thermal Cycler 9700 (Applied Biosystems) can be used as a thermal cycling device.

It is desired that in addition to the PCR thermal cycle function, a thermostat function at 50° C. or higher for keeping the aqueous solution of the gelling agent, the reaction solution, and the mixed oil at high temperatures during reaction setup should be imparted to the thermal cycling device.

(3) Process for Identification of Gelled Droplets which Contain Amplicon

After reaction, a 5-fold volume of isopropanol with respect to the emulsion is added thereto to prepare the emulsion in a form of one solution. The gelled droplet beads are collected by spin down.

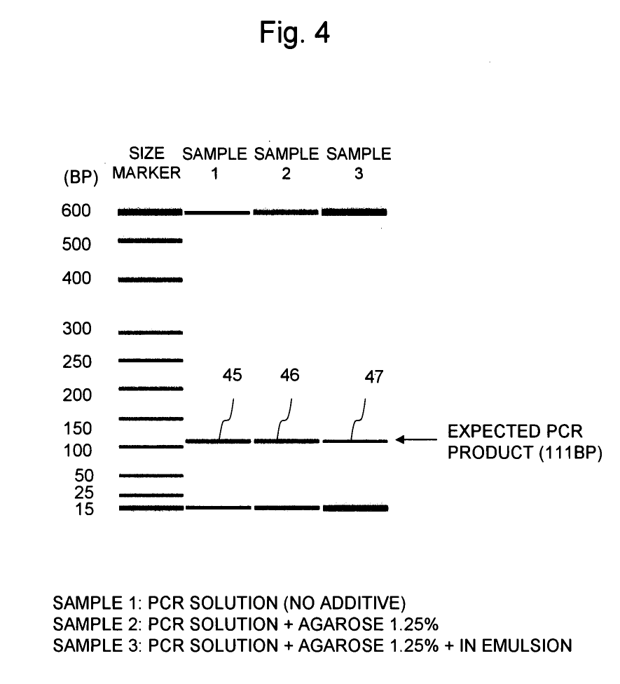

The collected gelled droplet beads which contain an amplicon can be subjected to gel electrophoresis (Agilent Bioanalyzer, DNA 500 kit or 2% agarose gel) to confirm the size and amount of the amplicon. FIG. 4 shows electrophoretic analysis results of amplicons from 1×10⁶ added template molecules using Agilent Bioanalyzer. A sample with no gelling agent (Sample 1) and a sample containing droplets in a non-emulsion state (Sample 2) were also prepared according to the same reaction solution composition and compared therewith.

A band 47 of the collected sample (Sample 3) was migrated to the same position (111 bp (base pair)) as a band 45 of the sample with no gelling agent (Sample 1) and a band 46 of the sample containing droplets in a non-emulsion state (Sample 2). As a result, the product with the same size as the comparative products could be confirmed to be formed.

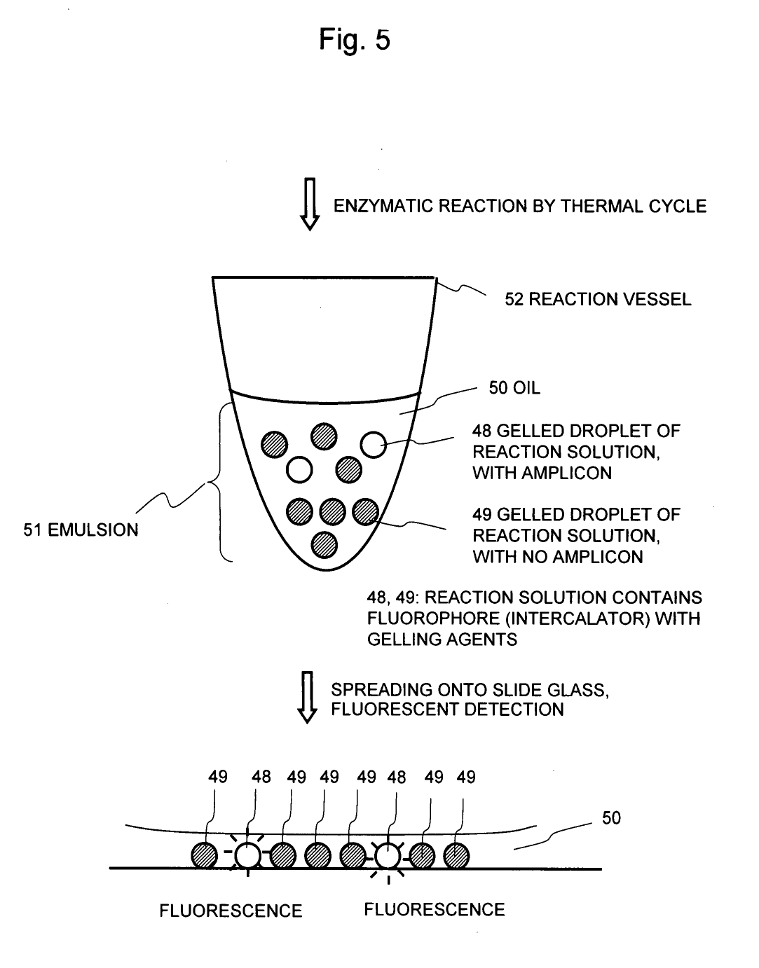

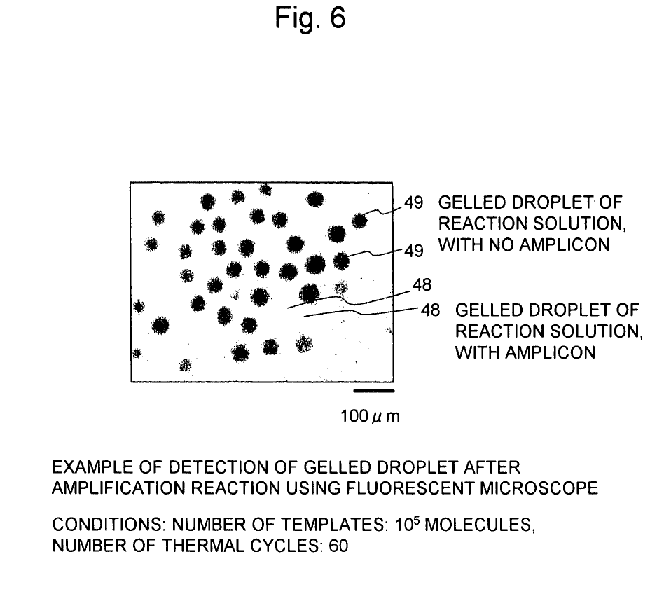

The reaction solution in an emulsion state after amplification can be observed directly with a fluorescent microscope (constitutional example: Olympus BX51, U1S-2 optical system, objective lens UplanSApo, mirror unit WIB-UMWIB3) without purification as described above. FIG. 5 schematically shows the observation state. FIG. 6 shows one example of fluorescent observation results of amplicons from 1×10⁵ added template molecules. Of gelled droplets 48 and 49 of the reaction solution, the droplet 48 with an amplicon is observed brightly by fluorescence from SYBR Green I, whereas the droplet 49 with no amplicon is observed darkly.

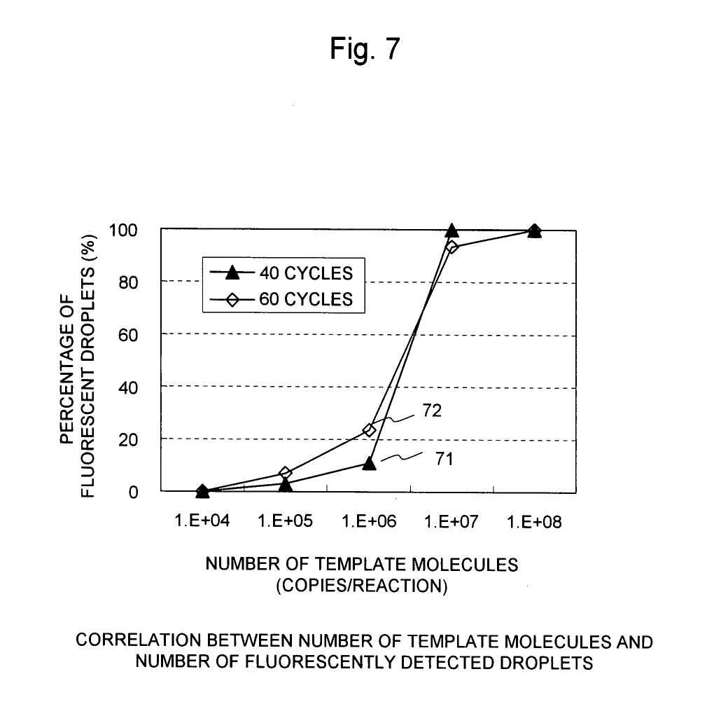

Observation is performed in the same manner as in FIGS. 5 and 6 by changing the number of templates per reaction. FIG. 7 shows a graph, wherein the percentage of the fluorescently detected droplet 48 with an amplicon is plotted in a line 71 for 40 thermal cycles and in a line 72 for 60 thermal cycles.

As shown in FIG. 7, the percentage hardly differs between the results of 40 thermal cycles and 60 thermal cycles, suggesting that the number of the droplets with an amplicon reaches a plateau in 40 cycles by efficient amplification.

Assuming that the droplets are 50 μm in average diameter, the average volume per droplet is 65 pl, and the number of the droplets per reaction (100 μl) is 1.5×10⁶. It is expected that when 10⁵ template molecules are added at the start of reaction, a little under 10% droplets contain one copy, and that when 10⁷ template molecules are added, almost all the droplets contain one or more copies of templates. The actual measurement results of the percentage of the detected droplets with an amplicon shown in FIG. 7 show values close to the expected values, wherein when 10⁵ template molecules are added, several % droplets with an amplicon are observed; when 10⁶ template molecules are added, dozen % droplets with an amplicon are observed; and when 10⁷ template molecules are added, almost 100% droplets with an amplicon are observed. These results demonstrate that amplification in this Example successfully proceeded.

Moreover, the amount of the amplicon was also investigated. The concentration of the band 47 of the 111-bp product in the electrophoretic analysis results of the collected amplicon (Sample 3) shown in FIG. 4 was quantified to be approximately 1 ng/μl (value quantified with Agilent Bioanalyzer 2100). This means that approximately 100 ng/100 μl/reaction of the amplicon was collected. 100 ng of 111-bp double-stranded DNA corresponds to 1.4 μM, 8×10¹¹ molecules.

An amplification rate was also investigated. As can be seen from the results shown in FIG. 7, when 10⁶ template molecules are added at the start of reaction, approximately 10% droplets with an amplicon is observed. Therefore, given that the number of droplets per 100 μl/reaction is 1.5×10⁶ from the assumption described above, the number of the droplets with an amplicon is 10% thereof, that is, 1.5×10⁵. Thus, the number of PCR products per droplet with an amplicon is approximately 5×10⁶ molecules. This indicates that the amplification rate is as favorable as 5×10⁶ folds. In addition to this approach, a flow cytometry, which will be described later, may be used in amplicon observation.

(4) Process for Separation of Gelled Droplets which Contain Amplicon

In this Example, the gelled droplets which contain an amplicon were collected with a pipette equipped with a capillary tube (e.g., Sequencing pipette manufactured by Drummond) under microscopic observation.

The amount of the amplicon contained in the collected droplets could be quantified by real-time PCR. The amplicon may be subjected to amplification processes again and to sequencing using a Sanger or Pyrosequencing method.

In addition to this approach, a flow cytometry, which will be described later, is also applicable to a collection method. According to this Example, a large number (10⁶) of samples in small amounts can be amplified up to 5×10⁶ folds simultaneously and individually by PCR, and the obtained amplicons can be identified on the basis of fluorescence and collected as gelled droplets. This individual collection does not require providing a solid phase in a reaction solution. Therefore, cost and labors for this purpose are saved. Moreover, reduction in reaction efficiency attributed to a solid phase can be prevented.

### Example 2

### Shape of Reaction Vessel

In this Example, the shape of a reaction vessel comprises a plate in which mutually separated small reaction cells are arranged.

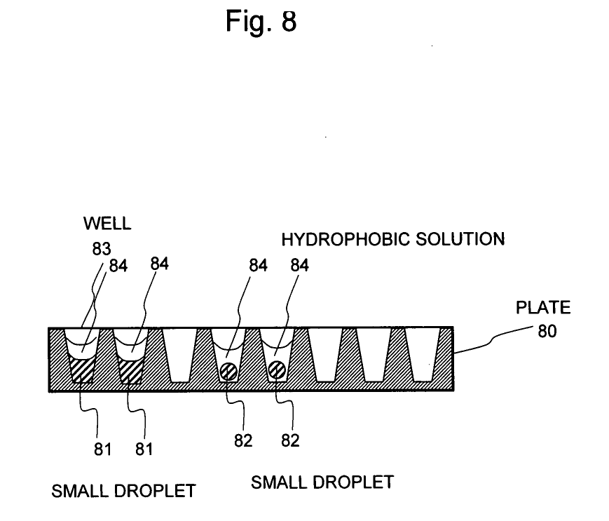

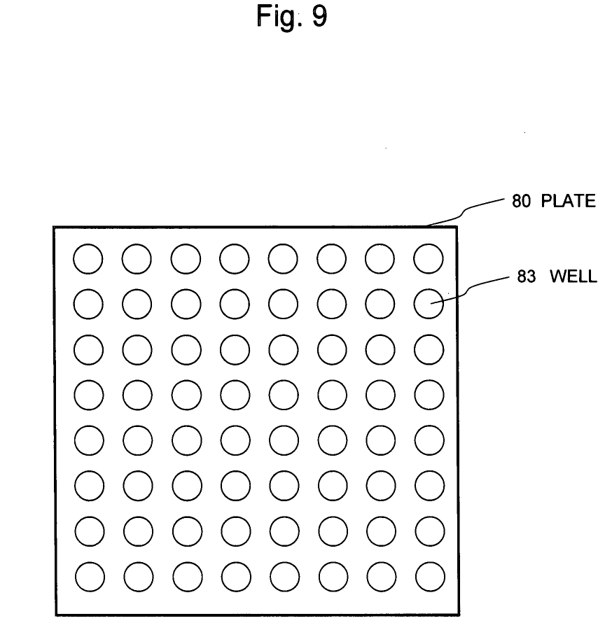

This Example will be described with reference to FIGS. 8 to 10. As shown in FIG. 8, a plate 80 is provided with a large number of wells 83 for accommodating individual small droplets 81 and 82. The wells 83 are two-dimensionally arranged, as shown in FIG. 9, to constitute the plate 80. The droplet may be contained directly in the well 83 and covered with a hydrophobic solution 84 or may be contained in the hydrophobic solution 84 in the well 83.

In this case, the hydrophobic solution 84 is used for the purpose of forming an emulsion and further functions to prevent water evaporation from the reaction solution, to keep the shape of the droplets spherical, and to prevent the adhesion between the gel and the vessel surface during the isolation of the gel.

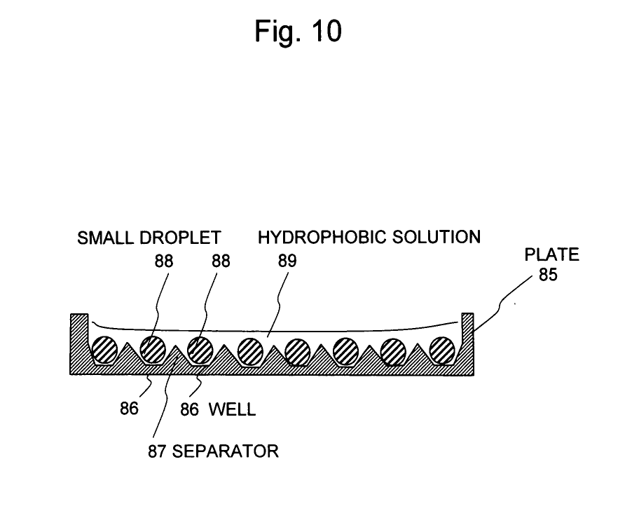

The droplets 81 and 82 must be separated mutually. However, the wells 83 themselves are not necessarily required to be mutually separated. As shown in a plate 85 of FIG. 10, the movement of droplets 88 may be restricted by a separator 87 between wells 86, and plural droplets 88 may be separated by a hydrophobic solution 89 that fills each well.

A preferable diameter of each well is 5 μm to 150 μm for the simultaneous amplification of a large number of samples. The number of wells is not particularly limited and is desirably 100,000 or more for the purpose of amplifying all expressed genes derived from one cell.

A preferable material of the plate is a heat-resistant clear plastic (e.g., polycarbonate) or glass for thermal cycles and optical measurement.

According to this Example, the droplets after reaction are spread on the flat surface of a plate. Therefore, observation after amplification is easily performed. Moreover, the position of each droplet on the flat surface is fixed. Therefore, the droplet can be distinguished from the other droplets on the basis of the position thereof.

### Example 3

In this Example, another method for producing small droplets will be illustrated.

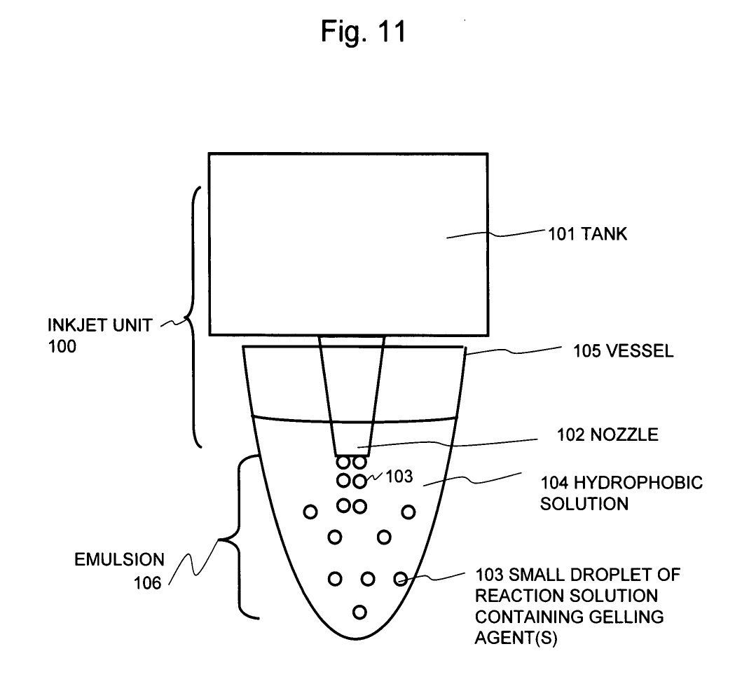

This Example will be described with reference to FIG. 11. In this Example, an ink jet unit 100 is used in droplet formation. The ink jet unit 100 comprises a tank 101 for storing a solution for preparation of droplets 103 and a nozzle 102 for spouting the formed droplets. The nozzle spouts a predetermined amount of a reaction solution by momentarily heating the reaction solution. The droplets 103 are placed in a vessel 105 so that the droplets 103 are directly spouted or allowed to fall into a hydrophobic solution 104. The droplets 103 are spouted or allowed to fall into the hydrophobic solution 104 to thereby prepare an emulsion 106.

This Example is suitable for controlling the size and quantity of the droplets and is particularly suitable for preparing approximately 0.5 pl to 10 pl droplets (approximately 10 μm to 30 μm in diameter). When the droplets are directly spouted into the hydrophobic solution, mutual sample contamination is effectively prevented.

### Example 4

This Example relates to constitution in which a flow cell is used in the detection and separation of small droplets with an amplicon.

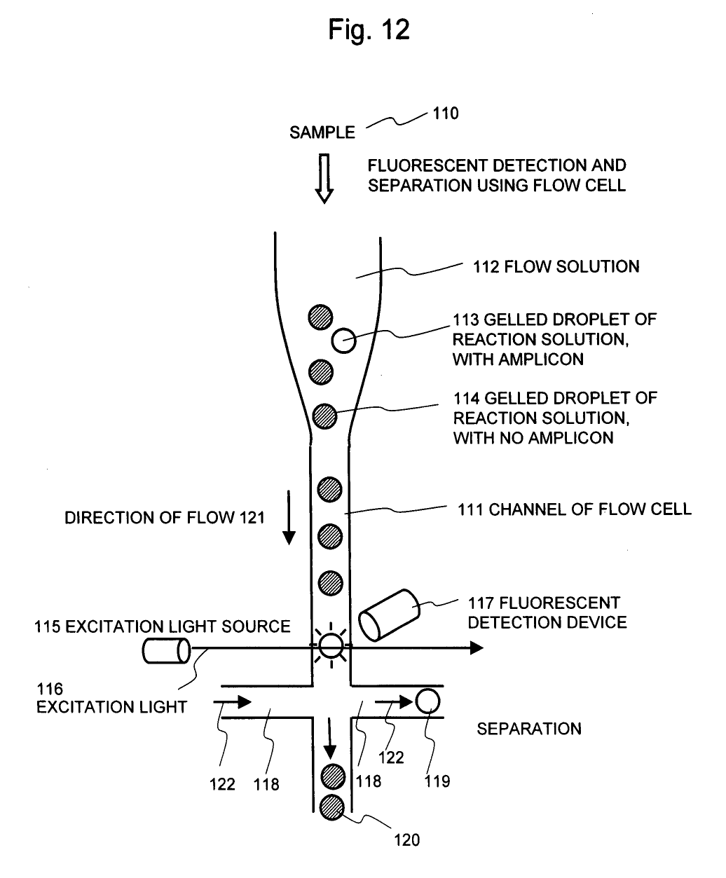

This Example will be described with reference to FIG. 12. A sample 110 containing small droplets 113 and 114 after amplification is poured along with a direction 121 of flow of a flow solution 112 into a channel 111 of a flow cell forming an optical cell. The sample may be poured thereinto by a free fall or with a pump. The droplets are irradiated with excitation light 116 from an excitation light source 115. The obtained fluorescence is detected with a fluorescent detection device 117 comprising a photodetector, a lens, a filter, and so on. The amount (or presence or absence) of an amplicon is determined on the basis of the obtained fluorescence intensity. A preferable flow solution 112 poured into the channel is silicone oil (e.g., Polyphenylmethylsiloxane) for the emulsion composition of Example 1.

A droplet 119 with fluorescence intensity larger than a predetermined level is separated from a droplet 120 with fluorescence intensity smaller than a predetermined level by causing a flow 122 of another channel 118. Then, this droplet 119 is collected. The droplet with fluorescence intensity smaller than a predetermined level may be separated and collected in the same way. To collect the droplet into another channel 118, the gel of the droplet 119 may be dissolved by local heating with a laser or the like and then collected.

According to this Example, the procedure of separating and collecting droplets after amplification according to amplicon contents thereof can be performed continuously and automatically.

### Example 5

In this Example, an apparatus for performing the method of the present invention will be described.

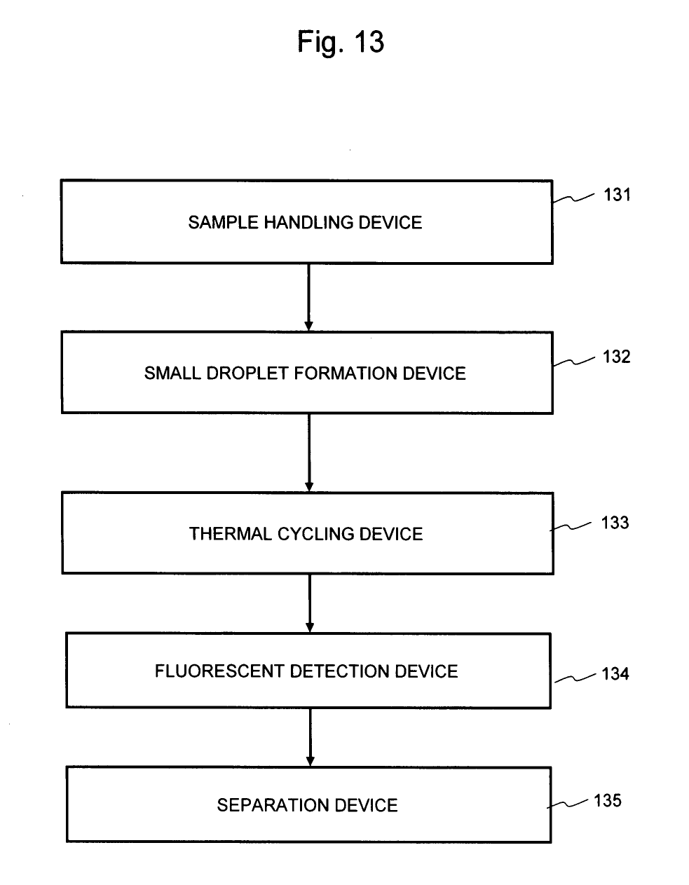

FIG. 13 shows a block diagram of the apparatus. The apparatus of this Example comprises a sample handling device 131, a small droplet formation device 132, a thermal cycling device 133, a fluorescent detection device 134, and a separation device 135.

The sample handling device 131 is equipped with a temperature control device for storing a gelling agent in a solution state, a liquid handling device for mixing the gelling agent and a reaction solution, and a stirring device. The temperature control device controls a temperature within a range of 0 to 120° C., which corresponds to a temperature necessary for the rapid dissolution of the gelling agent.

The small droplet formation device 132 comprises a stirring device comprising any of an oscillating or rotating mixer, the ink jet described in Example 3, and the method using microfluidics.

The thermal cycling device 133 is equipped with the same temperature control device as in a general PCR thermal cycler. Its temperature control device may also serve as that of the sample handling device 131.

The fluorescent detection device 134 comprises a fluorescent microscopic imaging or flow-cell detector.

The separation device 135 is equipped with a channel switching device provided along with the flow cell, as described in Example 4.

According to this Example, a large number of samples in small amounts can be amplified individually and simultaneously by PCR, and the obtained amplicons can be identified on the basis of fluorescence without performing the step of collecting gelled droplets.

The present invention provides an elemental technique necessary for quantitative analysis conducted by the digital counting of all mRNAs contained in one cell or a plurality of mRNAs probably in need of measurement. Thus, the present invention is useful in every field including biological, medical, and chemical fields and other fields that require single molecule analysis.

### [Free Text of Sequence Listing]

SEQ ID NO:1: Primer

SEQ ID NO: 2: Primer

What is claimed is:

1. A method for individually amplifying and isolating a plurality of nucleic acids in a sample, comprising subjecting the sample diluted so that the number of the nucleic acid contained in one droplet does not exceed one to PCR in the droplets in a hydrophobic solution and separating the reaction solution in a solid or gel state after the completion of PCR.

2. The method according to claim 1, further comprising the step of adding in advance a fluorophore capable of binding to or intercalating into an amplicon to the PCR reaction solution and thereby selecting and separating only the droplet containing the amplicon.

3. The method according to claim 1, wherein the PCR is performed in an emulsion of the droplets dispersed in the hydrophobic solution.

4. The method according to claim 1, wherein the PCR is performed in mutually separated small reaction cells arranged in a plate.

5. The method according to claim 1, wherein an adaptor sequence is introduced in advance in each of the plurality of nucleic acids in a sample so as to amplify the plurality of nucleic acids with a single PCR primer.

6. The method according to claim 1, wherein any one gelling agent selected from agarose, gelatin, starch (amylose), carrageenan, pectin, agaropectin, polyacrylamide, polyacrylic acid, polyvinyl alcohol, and polyvinylpyrrolidone is added in advance to the PCR reaction solution for separating the reaction solution in a solid or gel state.

7. The method according to claim 1, wherein the hydrophobic solution is mainly composed of silicone oil or paraffin oil.

8. The method according to claim 1, wherein a surfactant and/or a coating agent are further added in advance to the PCR reaction solution.

9. A method for nucleic acid analysis comprising the step of detecting or quantifying a plurality of nucleic acids individually amplified and isolated by a method according to claim 1.

10. An apparatus for individually amplifying and isolating a plurality of nucleic acids, comprising: 1) a sample handling device comprising a temperature control device for storing a gelling agent in a solution state, a liquid handling device for mixing the gelling agent and a reaction solution, and a stirring device; 2) a droplet formation device comprising any of an oscillating or rotating mixer, an ink jet, and microfluidics; 3) a temperature control device having a thermal cycle function for PCR; and 4) a fluorescent detection device equipped with an imaging or flow-cell detector.

11. The apparatus according to claim 10, wherein the flow cell in the fluorescent detection device 4) has a separation function by channel switching.

12. The apparatus according to claim 10 further comprising a DNA sequencer and/or a flow cytometry.

13. The apparatus according to claim 12, wherein the PCR is performed in an emulsion of the droplets dispersed in the hydrophobic solution.

14. The apparatus according to claim 12, wherein the PCR is performed in mutually separated small reaction cells arranged in a plate.

15. The apparatus according to claim 12, wherein an adaptor sequence is introduced in advance in each of the plurality of nucleic acids in a sample so as to amplify the plurality of nucleic acids with a single PCR primer.

16. The apparatus according to claim 12, wherein any one gelling agent selected from agarose, gelatin, starch (amylose), carrageenan, pectin, agaropectin, polyacrylamide, polyacrylic acid, polyvinyl alcohol, and polyvinylpyrrolidone is added in advance to the PCR reaction solution for separating the reaction solution in a solid or gel state.

17. The apparatus according to claim 12, wherein the hydrophobic solution is mainly composed of silicone oil or paraffin oil.

18. The apparatus according to claim 12, wherein a surfactant and/or a coating agent are further added in advance to the PCR reaction solution.

19. The apparatus according to claim 12, wherein the flow cell in the fluorescent detection device 4) has a separation function by channel switching.


SEQUENCE LISTING (2 sequences)

SEQ ID NO: 1 - DNA, 21 bp, Artificial sequence
  chemically synthesized
gttttcccagtcacgacgttg

SEQ ID NO: 2 - DNA, 21 bp, Artificial sequence
  chemically synthesized
atgaccatgattacgccaagc